(19) United States
(12) Patent Application Publication
(10) Pub. No.: US 2009/0169583 A1
(43) Pub. Date: Jul. 2, 2009

(54) Solid Adsorbates of Hydrophobic Drugs

(75) Inventors: Timothy James Brodeur, Bend, OR (US); Daniel Tod Smithey, Bend, OR (US); Ralph Tadday, Bend, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(73) Assignee: Pfizer, Inc.

(21) Appl. No.: 11/795,743

(22) PCT Filed: Jan. 30, 2006
(86) PCT No.: PCT/IB06/00337

§ 371 Date: Nov. 21, 2007

Related U.S. Application Data
(60) Provisional application No. 60/651,352, filed on Feb. 8, 2005.

(51) Int. Cl. A61K 9/00; A61K 31/47; A61K 31/40

(52) U.S. Cl. 424/400; 514/311; 514/423

(57) ABSTRACT

A solid pharmaceutical composition comprises a solid adsorbate comprising a hydrophobic drug, a lipophilic vehicle, and a porous substrate, wherein the hydrophobic drug and lipophilic vehicle are adsorbed to the porous substrate.

### BACKGROUND OF THE INVENTION

The present invention relates to a solid pharmaceutical composition comprising a solid adsorbate comprising a hydrophobic drug and a lipophilic vehicle adsorbed onto a porous solid substrate.

Hydrophobic drugs often show poor bioavailability or irregular absorption, the degree of irregularity being affected by factors such as dose level, fed state of the patient, and form of the drug.

Increasing the bioavailability of hydrophobic drugs has been the subject of much research. Increasing bioavailability hinges on improving the concentration of the drug in solution to improve absorption.

Due to their low aqueous solubility and hydrophobic character, hydrophobic drugs have generally proven to be difficult to formulate for oral administration such that high bioavailabilities are achieved. The dosage form must contain one or more excipients capable of improving either the dissolution rate of the hydrophobic drug, the amount of hydrophobic drug dissolved in the aqueous environment of the GI tract, or both. The mass of an orally administered dosage form is preferably 1 gram or less. Since the mass of the hydrophobic drug may range from 1 mg to 400 mg or more in the dosage form, the ratio of the mass of hydrophobic drug to formulation excipients (sometimes referred to as drug loading) should be high enough to allow preparation of oral dosage forms with a mass of 1 gram or less.

One approach to the delivery of hydrophobic drugs is to dissolve the drug in an oil or other vehicle, which is then administered to the patient. Because of the nature of the vehicle, it is often difficult to formulate such compositions into a solid dosage form suitable for oral delivery, such as a compressed tablet or pill.

Pather et al., U.S. Pat. No. 6,280,770, disclose so-called drug “microemulsions” adsorbed onto solid particulate adsorbents. The liquid microemulsion can be adsorbed onto the solid particulate adsorbent by the use of a planetary mixer, a Z-blade mixer, a rotorgranulator or similar equipment. Pather et al. state that preferably, the amount of microemulsion is kept sufficiently low so that the mixture of adsorbent and microemulsion forms an easily compressible, free-flowing powder. Pather et al. exemplify solid compositions in which the amount of drug present in the solid composition is quite low—less than 1 wt % in the examples disclosed.

Liu et al., U.S. Pat. No. 6,316,497, disclose a stabilized self-emulsifying system comprising anticancer medicament. The stabilized self-emulsifying system comprises a therapeutically effective amount of o-(chloroacetylcarbamoyl) fumigillol, a pharmaceutically acceptable carrier, and a stabilizing component, wherein the pharmaceutically acceptable carrier comprises an oily constituent and at least one surfactant. Liu et al. state that in one embodiment, the stabilizing agent may be suitable adsorbents or complex forming agents selected from the group consisting of gelatin, active charcoal, silica gel, and chelating agents. The pharmaceutically acceptable carrier having the medicament can be filled, mixed, adsorbed, filtered or otherwise combined, contacted, or reacted with the adsorbent or complex forming agent. According to Liu et al., the adsorbent or complex-forming agent typically comprises from about 0.05% to 15% weight adsorbent or complex-forming agent relative to the weight of the medicament.

What is still desired is a solid composition with high drug loading that provides enhanced dissolution and/or bioavailability of hydrophobic drugs, and can be formulated into solid dosage forms. These needs and others that will become apparent to one of ordinary skill are met by the present invention, which is summarized and described in detail below.

### BRIEF SUMMARY OF THE INVENTION

The present invention overcomes the drawbacks of the prior art by providing a solid adsorbate comprising a hydrophobic drug, a lipophilic vehicle, and a porous substrate, wherein the hydrophobic drug and lipophilic vehicle are adsorbed onto the porous substrate. The solid adsorbate provides enhanced dissolution and/or bioavailability of the hydrophobic drug.

In another aspect, the invention provides a dosage form comprising a solid adsorbate comprising a hydrophobic drug, a lipophilic vehicle, and a porous substrate, wherein the hydrophobic drug and lipophilic vehicle are adsorbed to the porous substrate.

In one embodiment, the hydrophobic drug is a cholesteryl ester transfer protein (CETP) inhibitor. In another embodiment, the hydrophobic drug is a cholesteryl ester transfer protein (CETP) inhibitor and the dosage form further comprises an HMG-CoA reductase inhibitor.

In yet another aspect, the invention provides a method for forming a solid adsorbate, comprising (a) forming a suspension or slurry comprising a hydrophobic drug, a lipophilic vehicle, a porous substrate, and a volatile solvent; and (b) removing at least a portion of the volatile solvent from the suspension or slurry so as to form the solid adsorbate, wherein the solid adsorbate comprises the hydrophobic drug and the lipophilic vehicle adsorbed to the porous substrate.

The inventors recognized and solved the problem of both low bioavailability and low drug loading for a class of hydrophobic drugs. The inventors found that relatively large amounts of the hydrophobic drug may be adsorbed onto the porous substrate, resulting in drug loadings of greater than 2 wt % of the adsorbate. The solid adsorbates, while containing a lipophilic vehicle, nonetheless remain solid and may be easily incorporated into a solid dosage form such as a tablet.

In addition, the inventors found that combining the hydrophobic drug with a lipophilic vehicle and then adsorbing this mixture onto a porous substrate, results in a composition that provides an enhanced dissolved concentration of the drug in an aqueous use environment. Without wishing to be bound by any particular theory, it is believed that upon administration of such compositions to an aqueous use environment, a microemulsion comprising the drug and the lipophilic vehicle is formed. This microemulsion provides enhanced concentration and/or bioavailability in in vivo aqueous use environments.

Furthermore, because the compositions of the present invention provide a higher concentration of drug dissolved in the use environment, and because once a high drug concentration is achieved the concentration tends to remain high due to solubilization of the drug in surfactant-stabilized oil droplets, the compositions may have a number of positive effects. First, in cases where the use environment is the GI tract, due to a prolonged high drug concentration, absorption of drug may continue over a longer time period and an effective concentration of drug in the blood may be maintained over a longer time period. Second, the compositions of the present invention may show less variability in drug absorption as a result of variation in the fed/fasted state of the GI tract of the patient.

It is believed that the compositions of the present invention, when administered to an aqueous use environment, such as the GI tract, form a plurality of small emulsion droplets comprising the drug and the lipophilic vehicle. These emulsion droplets are capable of sufficiently solubilizing the drug in the use environment to enhance bioavailability. When the lipophilic droplets are small, their high mobility may also increase the rate of drug absorption in the intestines by increasing the transport rate of the drug through the unstirred boundary layer adjacent to the intestinal wall. In combination, these properties may greatly enhance the rate and extent of drug absorption (e.g., bioavailability). The majority of water soluble drugs after absorption into the enterocytes of the intestine are transported into the portal vein via the process of diffusion. However, highly lipophilic (hydrophobic; Log P>4) drugs may also associate with lymph lipoproteins in the enterocyte and consequently get transported through the mesenteric lymphatic ducts, bypassing the liver and gain access into systemic circulation. The fractional amount of drug transported via the two pathways from the enterocyte may be influenced by not only the lipophilicity of the drug but also by the formulation components. The inclusion of lipophilic excipients such as fatty acids, mono, di and triglycerides etc. that are absorbed via the pathways of lipid digestion and lipid absorption can significantly promote the lymphatic absorption of lipophilic drugs. Extremely high concentrations of lipophilic drugs can be achieved in the lymph and it provides advantages for drug delivery, especially for those molecules that may undergo first pass liver metabolism. See for example, Adv. Drug Delivery Reviews, 50, 3-20 (2001).

In addition, the compositions may also have the advantage of providing more regular absorption between the fed and fasted state of a patient. It is well known in the art that in the fed state, the concentration of bile-salt micelles present in the GI tract is greater than the concentration present in the fasted state. It is believed that drug can readily partition into such bile-salt micelles, and drug in bile-salt micelles is readily absorbable because it is labile and the micelles are highly mobile. The inventors believe that this difference in the concentration of bile-salt micelles in the GI tract in the fed versus fasted state may account, at least in part, for the fed/fasted differences in bioavailability observed for many pharmaceutical compositions. The small emulsion droplets formed when the compositions of the present invention are administered to an aqueous use environment are believed to behave in a similar way as bile-salt micelles, thus providing a more uniform amount of drug in highly labile, highly mobile species between the fed and fasted state, resulting in a more uniform bioavailability between the fed and fasted state.

The foregoing and other objectives, features, and advantages of the invention will be more readily understood upon consideration of the following detailed description of the invention.

### DETAILED DESCRIPTION OF THE PREFERRED EMBODIMENTS

The present invention provides, in one aspect, a solid adsorbate comprising a hydrophobic drug and a lipophilic vehicle, wherein the hydrophobic drug and the lipophilic vehicle are adsorbed to a porous substrate. The composition provides enhanced concentration of the drug upon administration to an aqueous use environment.

In another aspect, the invention provides a dosage form comprising a solid adsorbate comprising a hydrophobic drug and a lipophilic vehicle, wherein the hydrophobic drug and lipophilic vehicle are adsorbed to a porous substrate. In one embodiment, the hydrophobic drug is a CETP inhibitor. In another embodiment, the hydrophobic drug is a CETP inhibitor and the dosage form further comprises an HMG-CoA reductase inhibitor.

Reference to an “aqueous use environment” can either mean in vivo fluids, such as the GI tract, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human, or the in vitro environment of a test solution, such as phosphate buffered saline (PBS), a Model Fasted Duodenal (MFD) solution, or a solution to model the fed state. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. An appropriate MFD solution is the same PBS solution wherein additionally is present 7.3 mM sodium taurocholic acid and 1.4 mM of 1-palmitoyl-2-oleyl-sn-glycero-3-phosphocholine. An appropriate solution to model the fed state is the same PBS solution wherein additionally is present 29.2 mM sodium taurocholic acid and 5.6 mM of 1-palmitoyl-2-oleyl-sn-glycero-3-phosphocholine.

“Administration” to a use environment means, where the in vivo use environment is the GI tract, delivery by ingestion or swallowing or other such means to deliver the drug. One skilled in the art will understand that “administration” to other in vivo use environments means contacting the use environment with the composition of the invention using methods known in the art. See for example, Remington: The Science and Practice of Pharmacy, 20^{th} Edition (2000). Where the use environment is in vitro, “administration” refers to placement or delivery of the composition in the in vitro test medium. Where release of drug into the stomach is not desired but release of the drug in the duodenum or small intestine is desired, the use environment may also be the duodenum or small intestine. In such cases, “introduction” to a use environment is that point in time when the dosage form leaves the stomach and enters the duodenum.

Hydrophobic drugs, lipophilic vehicles, porous substrates, methods for making solid adsorbates, suitable excipients and dosage forms are discussed in more detail below.

### Hydrophobic Drugs

The term “drug” is conventional, denoting a compound having beneficial prophylactic and/or therapeutic properties when administered to an animal, especially humans. The drug may be in any pharmaceutically acceptable form. By “pharmaceutically acceptable form” is meant any pharmaceutically acceptable derivative or variation, including stereoisomers, stereoisomer mixtures, enantiomers, tautomers, solvates, hydrates, isomorphs, polymorphs, pseudomorphs, neutral forms, salt forms and prodrugs.

The relative degree of enhancement in aqueous concentration and bioavailability provided by the compositions of the present invention generally improves for drugs as solubility decreases and hydrophobicity increases. In fact, the inventors have recognized a subclass of drugs that are essentially aqueous insoluble, highly hydrophobic, and are characterized by a set of physical properties. This subclass, referred to herein as “hydrophobic drugs,” exhibits dramatic enhancements in aqueous concentration and bioavailability when formulated in the compositions of the present invention.

The first property of hydrophobic drugs is that the Log P value of the drug may have a value of at least 4.0, a value of at least 4.5, or even a value of at least 5.0. Log P, defined as the base 10 logarithm of the ratio of (1) the drug concentration in an octenol phase to (2) the drug concentration in a water phase when the two phases are in equilibrium with each other, is a widely accepted measure of hydrophobicity. Log P may be measured experimentally or calculated using methods known in the art. The Log P may be estimated experimentally by determining the ratio of the drug solubility in octanol to the drug solubility in water. When using a calculated value for Log P, the highest value calculated using any generally accepted method for calculating Log P is used. Calculated Log P values are often referred to by the calculation method, such as Clog P, Alog P, and Mlog P. The Log P may also be estimated using fragmentation methods, such as Crippen's fragmentation method (J. Chem. Inf. Comput. Sci., 27, 21 (1987)); Viswanadhan's fragmentation method (J. Chem. Inf. Comput. Sci., 29, 163 (1989)); or Broto's fragmentation method (Eur. J. Med. Chem.-Chlm. Theor., 19, 71 (1984). Preferably the Log P value is calculated by using the average value estimated using Crippen's, Viswanadhan's, and Broto's fragmentation methods.

A second property of hydrophobic drugs is that they have low aqueous solubility. By low aqueous solubility is meant that the minimum aqueous solubility at physiologically relevant pH (pH of 1 to 8) at about 22° C. is less than about 100 μg/ml and often less than about 10 μg/ml. (Unless otherwise specified, reference to aqueous solubility herein and in the claims is determined at about 22° C.) in addition, hydrophobic drugs often have a very high dose-to-solubility ratio. Extremely low aqueous solubility often leads to poor or slow absorption of the drug from the fluid of the gastrointestinal tract, when the drug is dosed orally in a conventional manner. For extremely low solubility drugs, absorption generally becomes progressively more difficult as the dose (mass of drug given orally) increases. Thus, a third property of hydrophobic drugs is a very high dose (in mg) to solubility (In mg/ml) ratio (ml). By “very high dose-to-solubility ratio” is meant that the dose-to-solubility ratio may have a value of at least 1000 ml, at least 5,000 ml, or even at least 10,000 ml. The dose-to-solubility ratio may be determined by dividing the dose (in mg) by the aqueous solubility (in mg/ml).

Hydrophobic drugs also typically have very low absolute bioavailabilities. Specifically, the absolute bioavailability of hydrophobic drugs, when dosed orally in their unformulated state (i.e., drug alone) is typically less than about 10% and more often less than about 5%.

The invention finds particular utility for drugs that are soluble in the lipophilic vehicle, but which nonetheless do not aggregate in the aqueous use environment to form a single phase. Solubility of the hydrophobic drug in the lipophilic vehicle, which can consist of one or more components, is desirable because it reduces the amount of lipophilic vehicle that must be present in the composition to achieve a given dose of hydrophobic drug, thus increasing the weight fraction of drug present in the composition. In general, hydrophobic drugs with Log P values in the range of from about 4 to about 10 have moderate to high solubility in the lipophilic vehicle. Since the invention finds greater utility with increasing solubility of the drug in the lipophilic vehicle, the Log P value of the hydrophobic drug may be greater than about 4.5, or even greater than about 5. However, if the Log P value of the hydrophobic drug is too high, the composition may not be effective. At high Log P values, the hydrophobic drug may not be released from the lipophilic vehicle when introduced to an aqueous environment of use, resulting in poor concentration enhancement and/or bioavailability. Alternatively, the high Log P value of the drug may result in lower solubility of the drug in the lipophilic vehicle. Thus, in one embodiment, the Log P value of the hydrophobic drug ranges from about 4 to about 10. The invention has greater utility over the Log P range of from about 4.5 to about 9, and even greater utility over the range of from about 5 to about 8.

The solubility of the drug in the lipophilic vehicle is also a function of the melting point (Tₘ) of the drug. In general, for a given Log P value, the solubility of the drug in the lipophilic vehicle decreases with increasing melting point of the drug. For example, for two drugs each having a Log P of 6.5, one with a Tₘ value of about 80° C. and the other drug with a Tₘ value of about 120° C., the drug with the lower Tₘ will have a higher solubility in the lipophilic vehicle relative to the drug with the higher Tₘ. Moderate to high solubility of the hydrophobic drug in the lipophilic vehicle is generally obtained when the hydrophobic drug has a melting point of less than about 170° C. Since the invention finds increasing utility as the solubility of the lipophilic vehicle increases for a given Log P, the hydrophobic drug may have a melting point of less than about 150° C., or even less than about 140° C.

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol-reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, and antiviral agents, glycogen phosphorylase inhibitors, and cholesteryl ester transfer protein (CETP) inhibitors.

One class of hydrophobic drugs that work well in the compositions of the present invention is CETP inhibitors. CETP inhibitors are a class of compounds that are capable of modulating levels of blood cholesterol, such as by raising high-density lipoprotein (HDL) cholesterol and lowering low-density lipoprotein (LDL) cholesterol. It is desired to use CETP inhibitors to lower certain plasma lipid levels, such as LDL-cholesterol and triglycerides and to elevate certain other plasma lipid levels, including HDL-cholesterol, and accordingly to treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans.

CETP inhibitors, particularly those that have high binding activity, are generally hydrophobic, have extremely low aqueous solubility and have low oral bioavailability when dosed conventionally. CETP inhibitors, when formulated in the compositions of the present invention, show dramatic improvements in bioavailability and concentration-enhancement relative to crystalline drug alone.

CETP inhibitors are typically “substantially water-insoluble,” which means that the CETP inhibitor has a minimum aqueous solubility of less than about 10 μg/ml at any physiologically relevant pH (e.g., pH 1-8) and at about 22° C. Compositions of the present invention find greater utility as the solubility of the CETP inhibitors decreases, and thus are preferred for CETP inhibitors with solubilities less then about 10 μg/mL, and even more preferred for CETP inhibitors with solubilities less than about 1 μg/mL. Many CETP inhibitors have even lower solubilities (some even less than 0.1 μg/mL), and require dramatic concentration enhancement to be sufficiently bioavailable upon oral dosing for effective plasma concentrations to be reached at practical doses.

The invention is not limited by any particular structure or group of CETP inhibitors. Rather, the invention has general applicability to hydrophobic CETP inhibitors as a class. Specific examples of hydrophobic cholesteryl ester transfer protein (CETP) inhibitors include [2R,4S]4-[(3,5-bis-trifluoromethyl-benzyl)-methoxycarbonyl-amino]2-ethyl-6-trifluoromethyl-3,4-dihydro-2H-quinoline-1-carboxylic acid ethyl ester (torcetrapib), [2R,4S]4-[acetyl-(3,5-bis-trifluoromethyl-benzyl)-amino]-2-ethyl-6-trifluoromethyl-3,4-dihydro-2H-quinoline-1-carboxylic acid isopropyl ester, [2R,4S]-4-[(3,5-Bis-trifluoromethyl-benzyl)-methoxycarbonyl-amino]-2-ethyl-6-trifluoromethyl-3,4-dihydro-2H-quinoline-1-carboxylic acid isopropyl ester, (2R)-3-[[3-(4-chloro-3-ethylphenoxy)phenyl][[3-(1,1,2,2-tetrafluoroethoxy)phenyl]methyl]amino]-1,1,1-trifluoro-2-propanol, (2R,4R,4aS)-4-[amino-(3,5-bis-(trifluoromethyl-phenyl)-methyl]-2-ethyl-6-(trifluoromethyl)-3,4-dihydroquinoline-1-carboxylic acid isopropyl ester, S-[2-([[1-(2-ethylbutyl)cyclohexyl]carbonyl]amino)phenyl]2-methylpropanethioate, trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid, trans-4-[[[2-[[[[3,5-bis(trifluoromethyl)phenyl]methyl](2-methyl-2H-tetrazol-5-yl)amino]methyl]-5-methyl-4-(trifluoromethyl)phenyl]ethylamino]methyl]-cyclohexaneacetic acid, the drugs disclosed in commonly owned U.S. patent application Ser. Nos. 09/918,127 and 10/066,091, both of which are incorporated herein by reference in their entireties for all purposes, and the drugs disclosed in the following patents and published applications: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; WO 9835937 A1; JP 11049743; WO 200018721; WO 200018723; WO 200018724; WO 200017164; WO 200017165; WO 200017166; WO 2004020393; EP 992496; and EP 987251, all of which are hereby incorporated by reference in their entireties for all purposes.

In a preferred embodiment, the hydrophobic drug is the CETP inhibitor [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, also known as torcetrapib. Torcetrapib is shown by the following Formula

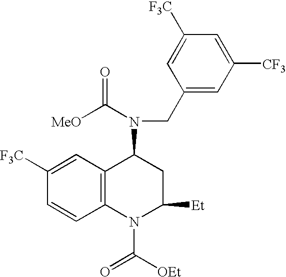

CETP inhibitors, in particular torcetrapib, and methods for preparing such compounds are disclosed in detail in U.S. Pat. Nos. 6,197,786 and 6,313,142, in PCT Application Nos. WO 01/40190A1, WO 02/088085A2, and WO 02/088069A2, the disclosures of which are herein incorporated by reference. Torcetrapib has an unusually low solubility in aqueous environments such as the lumenal fluid of the human GI tract. The aqueous solubility of torcetrapib is less than about 0.04 μg/ml. Torcetrapib must be presented to the GI tract in a solubility-enhanced form in order to achieve a sufficient drug concentration in the GI tract in order to achieve sufficient absorption into the blood to elicit the desired therapeutic effect.

### Lipophilic Vehicles

The compositions of the present invention also comprise a lipophilic vehicle. By “lipophilic vehicle” is meant any ingredient or combination of ingredients in which the hydrophobic drug may be dissolved. The lipophilic vehicle, which can comprise a single component or two or more components, should be (1) water immiscible, and (2) capable of forming a plurality of small lipophilic droplets when administered to the aqueous use environment. The lipophilic vehicle should also be pharmaceutically acceptable.

The lipophilic vehicle must be “water immiscible,” meaning that the material when administered as prescribed herein to an aqueous use environment exceeds its solubility as solvated molecules thus requiring the formation of a second phase. Ideally such a second phase takes the form of a large number of small phases such as micelles or a microemulsion. The lipophilic droplets are a separate phase in the aqueous use environment; the separate phase ranging from extremely small submicron sized aggregates such as micelles or as large droplets up to a few microns in size. The lipophilic vehicle should not agglomerate into a single phase within the use environment, but should remain as a plurality of droplets for at least 1 hour and preferably longer. It is to be understood that the lipophilic vehicle, when incorporated into the solid compositions of the present invention may or may not result in the formation of a microemulsion when the solid composition is administered to an aqueous use environment. However, the lipophilic vehicle alone, without any drug or solid substrate present, will result in the formation of an emulsion or microemulsion when administered to an aqueous use environment.

In a one embodiment, the lipophilic vehicle forms a self-emulsifying or self-microemulsifying composition. The term “self-emulsifying” refers to a formulation which, when diluted by a factor of at least 100 by water or other aqueous medium and gently mixed, yields an opaque, stable oil/water emulsion with a mean droplet diameter less than about 5 microns, but greater than about 100 nm, and which is generally polydisperse. Such an emulsion is stable for at least several (i.e., for at least 2) hours, meaning there is no visibly detectable phase separation and that there is no visibly detectable crystallization of hydrophobic drug.

The term “self-microemulsifying” refers to a formulation which, when diluted by a factor of at least 100 by water or other aqueous medium and gently mixed, yields a non-opaque, stable oil/water emulsion with a mean droplet diameter of about 1 micron or less, the mean droplet diameter preferably being less than 100 nm. Most preferably the emulsion is transparent and has a unimodal droplet diameter distribution with a mean diameter less than 50 nm as determined, for example, by dynamic light scattering. The microemulsion is thermodynamically stable and without any indication of crystallization of hydrophobic drug.

“Gentle mixing” as used above is understood in the art to refer to the formation of an emulsion by gentle hand (or machine) mixing, such as by repeated inversions on a standard laboratory mixing machine. High shear mixing is not required to form the emulsion. Such formulations generally emulsify nearly spontaneously when introduced into the human (or other animal) gastrointestinal tract.

The lipophilic vehicle may comprise an oil, a surfactant, a lipophilic solvent, or mixtures thereof. By “oil” is meant a material that (1) acts as a solvent for the hydrophobic drug, and (2) disperses in an aqueous use environment to form lipophilic phases. By “surfactant” is meant a material that has surface-active properties. Surfactants are generally amphiphilic materials, meaning that they have both hydrophilic and hydrophobic regions. Surfactants are often characterized by their “HLB” value, HLB being an acronym for “hydrophilic-lipophilic balance,” which ranges from 1 to 20. The higher the HLB value, the more hydrophilic the surfactant. Combinations of surfactants often provide superior performance. Thus, in one embodiment, the lipophilic vehicle comprises a mixture of at least one hydrophilic surfactant (HLB values of about 8 or more) and at least one hydrophobic surfactant (HLB values of about 8 or less). A mixture of surfactants is sometimes referred to in the art as a surfactant/cosurfactant system. By “lipophilic solvent” is meant a material in which the hydrophobic drug of interest is highly soluble, having, for any given hydrophobic drug, a solubility of at least 150 mg/mL. Lipophilic solvents are sometimes referred to in the art as cosolvents. Some materials may fall into two or all three of these broad classes of compounds.

The choice of lipophilic vehicle will depend on the physical/chemical properties of the drug and lipophilic vehicle components. The inventors have found that a suitable lipophilic vehicle for a particular drug can be identified by first matching the solubility parameters of the drug and lipophilic vehicle. Solubility parameters are a well-known tool in the art used to correlate and predict cohesive and adhesive properties of materials. A complete discussion of solubility parameters is provided in Barton's Handbook of Solubility Parameters and Other Cohesion Parameters (CRC Press, 1983, hereinafter referred to as “Barton”), which is hereby incorporated by reference.

While several methods can be used to determine the solubility parameter of a given compound, as used herein, by “solubility parameter” is meant the Hildebrand solubility parameter calculated from group molar cohesive energy constants, as described in Barton, pages 61 to 66.

Hildebrand solubility parameters have units of (J/cm³)^{1/2}.

In one method to identify a suitable lipophilic vehicle, the solubility parameters of the drug and candidate lipophilic vehicles are first determined, such as by using the group contribution methods described in Barton. A suitable lipophilic vehicle generally will typically have a solubility parameter that is within ±5 units of the solubility parameter of the drug. Once a candidate lipophilic vehicle has been identified by matching its solubility parameter with the drug, adjustments to the lipophilic vehicle can be made to improve the performance and stability of the composition using methods known to those skilled in the art.

Examples of oils suitable for use as the lipophilic vehicle include: medium-chain glyceryl mono-, di-, and tri-alkylates, such as mono and diglycerides of capric and caprylic acid (CAPMUL® MCM, MCM 8, and MCM 10, available commercially from Abitec, and IMWITOR® 988, 742 or 308, available commercially from Condea Vista), MYVEROL 18-92, ARLACEL 186, fractionated coconut oil (MIGLYOL 810, MIGLYOL 812, NEOBEE® M5, CAPTEX® 300, CAPTEX® 355, CRODAMOL® GTCC), light vegetable oils, triacetin; long chain glyceryl mono-, di-, and tri-alkylates, such as vegetable oils such as soybean, safflower, corn, olive, cottonseed, arachis, sunflower seed, palm, and rapeseed; sorbitan esters, such as ARLACEL 20, ARLACEL 40; long-chain fatty alcohols, such as stearyl alcohol, cetyl alcohol, cetostearyl alcohol; long-chain fatty-acids such as stearic acid; polyoxyethylene 6 apricot kernel oil, available under the registered trademark LABRAFIL® M 1944 CS from Gattefosse; polyoxyethylene corn oil, available commercially as LABRAFIL® M 2125; propylene glycol monolaurate, available commercially as Lauroglycol from Gattefosse; propylene glycol dicaprylate/caprate available commercially as CAPTEX® 200 from Abitec or MIGLYOL® 840 from Condea Vista; polyglyceryl oleate available commercially as PLUROL OLEIQUE from Gattefosse; sorbitan esters of fatty acids, such as SPAN® 20, CRILL® 1, CRILL® 4, available commercially from ICI and Croda; glyceryl monooleate, such as MAISINE and PECEOL); and mixtures thereof.

Examples of surfactants suitable for use as the lipophilic vehicle include: sulfonated hydrocarbons and their salts, such as sodium 1,4-bis(2-ethylhexyl) sulfosuccinate, also known as docusate sodium (CROPOL) and sodium lauryl sulfate (SLS); poloxamers, also referred to as polyoxyethylene-polyoxypropylene block copolymers (PLURONICS, LUTROLs); polyoxyethylene alkyl ethers (CREMOPHOR A, BRIJ); polyoxyethylene sorbitan fatty acid esters (polysorbates, TWEEN); short-chain glyceryl mono-alkylates (HODAG, IMWITTOR, MYRJ); polyglycolized glycerides (GELUCIREs); mono- and di-alkylate esters of polyols, such as glycerol; nonionic surfactants such as polyoxyethylene 20 sorbitan monooleate, (polysorbate 80, sold under the trademark TWEEN 80, available commercially from ICI); polyoxyethylene 20 sorbitan monolaurate (Polysorbate 20, TWEEN 20); polyethylene (40 or 60) hydrogenated castor oil (available under the trademarks CREMOPHOR® RH40 and RH60 from BASF); polyoxyethylene (35) castor oil (CREMOPHOR® EL); polyethylene (60) hydrogenated castor oil (Nikkol HCO-60); alpha tocopheryl polyethylene glycol 1000 succinate (Vitamin E TPGS); glyceryl PEG 8 caprylate/caprate (available commercially under the registered trademark LABRASOL® from Gattefosse); PEG 32 glyceryl laurate (sold commercially under the registered trademark GELUCIRE 44/14 by Gattefosse), polyoxyethylene fatty acid esters (available commercially under the registered trademark MYRJ from ICI), polyoxyethylene fatty acid ethers (available commercially under the registered trademark BRIJ from ICI).

Examples of lipophilic solvents include polyol esters of fatty acids, such as triacetin (1,2,3-propanetriol triacetate or glyceryl triacetate available from Eastman Chemical Corp.) and other trialkyl citrate esters; propylene carbonate; dimethylisosorbide; ethyl lactate; N-methyl pyrrolidones; transcutol; glycofurol; peppermint oil; 1,2-propylene glycol; polyethylene glycols; and mixtures thereof.

Additional examples of lipophilic vehicles suitable for use in the present invention are disclosed in commonly assigned, copending U.S. Patent Application No. 2003-0022944A1, the disclosure of which is incorporated herein by reference. See also Pharmaceutical Excipients 2003 (Pharmaceutical Press and American Pharmaceutical Association 2003), and U.S. Pat. No. 6,294,192 (B1), the disclosure of which is incorporated herein in its entirety by reference.

In one embodiment of the invention, the lipophilic vehicle comprises two or more materials selected from the group consisting of oils, surfactants, and lipophilic solvents.

### Porous Substrates

The hydrophobic drug and lipophilic vehicle are adsorbed to a water insoluble, porous substrate. The substrate may be any material that is inert, meaning that the substrate does not adversely interact with the drug to an unacceptably high degree and which is pharmaceutically acceptable. The substrate should be in the form of small particles ranging in size of from 5 nm to 1 μm, preferably ranging in size from 5 nm to 100 nm. These particles may in turn form agglomerates ranging in size from 10 nm to 100 μm. The substrate is also insoluble in the process environment used to form the composition of the invention.

The substrate also has a high surface area, meaning that the substrate has a surface area of at least 20 m²/g, preferably at least 50 m²/g, more preferably at least 100 m²/g, and most preferably at least 180 m²/g. The surface area of the substrate may be measured using standard procedures. One exemplary method is by low-temperature nitrogen adsorption, based on the Brunauer, Emmett, and Teller (BET) method, well known in the art. As discussed below, the higher the surface area of the substrate, the higher the drug-to-substrate ratio that can be achieved and still maintain high concentration-enhancements. Thus, effective substrates can have surface areas of up to 200 m²/g, up to 400 m²/g and up to 600 m²/g or more.

Exemplary materials which are suitable for the substrate include oxides, such as SiO₂, TiO₂, ZnO₂, ZnO, Al₂O₃, MgAlSilicate, calcium silicate (Zeodor™ and Zeopharm®), AlOH₂, magnesium oxide, magnesium trisilicate, silicon dioxide (Cab-O-Sil®) or Aerosil®), zeolites, and other inorganic molecular sieves; inorganic materials such as silica, fumed silica (such as Aeroperl® and Aerosil® from Degussa, Parsippany, N.J.), dibasic calcium phosphate, calcium carbonate magnesium hydroxide, and talc; clays, such as kaolin (hydrated aluminum silicate), bentonite (hydrated aluminum silicate), hectorite and Veegum®; Na-, Al-, and Fe-montmorillonite; water Insoluble polymers, such as cross-linked cellulose acetate phthalate, cross-linked hydroxypropyl methyl cellulose acetate succinate, cross-linked polyvinyl pyrrolidinone, (also known as cross povidone), microcrystalline cellulose, polyethylene/polyvinyl alcohol copolymer, polyethylene polyvinyl pyrrolidone copolymer, cross-linked carboxymethyl cellulose, sodium starch glycolate, cross-linked polystyrene divinyl benzene; and activated carbons, including those made by carbonization of polymers such as polyimides, polyacrylonitrile, phenolic resins, cellulose acetate, regenerated cellulose, and rayon. Highly porous materials such as calcium silicate and silicone dioxide are preferred.

The surface of the substrate may be modified with various substituents to achieve particular interactions of the drug or lipophilic vehicle ingredients with the substrate. For example, the substrate may have a hydrophobic or hydrophilic surface. By varying the terminating groups of substituents attached to the substrate, the interaction of ingredients with the substrate may be influenced. For example, it may be desired to select a substrate having hydrophobic substituents to improve the binding of the drug and lipophilic vehicle to the substrate.

### Preparation of Compositions

The solid adsorbates of the present invention comprise a hydrophobic drug and a lipophilic vehicle, wherein the hydrophobic drug and lipophilic vehicle are adsorbed to a porous substrate. The mass ratio of hydrophobic drug to lipophilic vehicle will depend on the properties of the hydrophobic drug and the lipophilic vehicle. Generally, the ratio of hydrophobic drug to lipophilic vehicle will range from about 0.01 (about 1 wt % hydrophobic drug) to about 4 (about 80 wt % hydrophobic drug). Preferably, the ratio of hydrophobic drug to lipophilic vehicle is at least about 0.05 (about 5 wt % hydrophobic drug), more preferably at least about 0.1 (about 9 wt % hydrophobic drug), Higher ratios of hydrophobic drug are desirable because they reduce the amount of lipophilic vehicle that must be present in the composition to achieve a given dose of hydrophobic drug, thus increasing the weight fraction of drug in the composition.

However, at very high ratios of hydrophobic drug to lipophilic vehicle, there is insufficient lipophilic vehicle present for the composition to result in increased bioavailability. Thus, it is also preferred that the ratio of hydrophobic drug to lipophilic vehicle be less than about 3 (75 wt % hydrophobic drug), more preferably less than about 2 (about 67 wt % hydrophobic drug), and most preferably less than about 1 (about 50 wt % hydrophobic drug). The inventors have found that hydrophobic drug to lipophilic vehicle ratios ranging from about 0.1 (about 9 wt % hydrophobic drug) to about 1 (about 50 wt % hydrophobic drug) perform well.

Further details on liquid self-emulsifying formulations are disclosed in commonly assigned copending U.S. Patent Application Number 20030022944A1 filed on Jun. 19, 2002, which is incorporated in its entirety by reference.

The hydrophobic drug in the composition is generally non-crystalline in nature, as measured using standard quantitative techniques, such as powder X ray diffraction (PXRD).

The hydrophobic drug and lipophilic vehicle are adsorbed to a porous substrate to form a solid adsorbate. As used herein, the term “solid” means that the adsorbate is a dry, noncohesive mixture that is generally processable using solids handling equipment well known in the art. The ratio of hydrophobic drug/lipophilic vehicle to porous substrate generally should be sufficiently low that the composition is solid. The inventors have found that preferably the porous substrate constitutes at least about 10 wt %, more preferably at least 15 wt %, and even more preferably at least 20 wt % of the solid adsorbate. Lower amounts of the porous substrate, (and thus higher amounts hydrophobic drug/lipophilic vehicle) often result in materials that have poor flow properties and are difficult to process.

In one embodiment, the solid adsorbate comprising the hydrophobic drug, lipophilic vehicle, and porous substrate is substantially free of water. As used herein, the term “substantially free of water” means that the composition, prior to administration to an aqueous use environment, contains less than about 10 wt % water based on the total weight of the composition. Preferably the composition contains less than about 5 wt % water, and more preferably less than about 1 wt % water.

The hydrophobic drug/lipophilic vehicle may be combined with the porous substrate to form the compositions of the present invention using any method that results in a solid adsorbate. When the hydrophobic drug/lipophilic vehicle combination is a liquid, the liquid may be adsorbed onto the porous substrate by the use of a planetary mixer, a Z-blade mixer, a rotorgranulator or similar equipment. Heat may be used to melt a portion of the hydrophobic drug/lipophilic vehicle in order to first form a liquid, which may then be combined with the porous substrate to form the solid composition. Excipients may also be included in the composition to reduce the temperature at which the composition becomes a liquid.

The solid adsorbate may also be formed in an extruder, such as a single screw or twin-screw extruder, well known in the art. Here, the hydrophobic drug, lipophilic vehicle, and porous substrate may be fed to the extruder, and heat and/or compression and/or shear forces may be used to adsorb the hydrophobic drug/lipophilic vehicle to the porous substrate.

Another method for forming the solid adsorbate of the present invention is to combine the hydrophobic drug, lipophilic vehicle, and porous substrate with a volatile solvent to form a suspension or slurry, and then remove at least a portion of the volatile solvent to form the solid composition. By “volatile solvent” is meant a compound or mixture of compounds having a boiling point of about 150° C. or less. Preferably the volatile solvent has a boiling point of about 120° C. less, and more preferably about 100° C. or less. The volatile solvent can be any compound or mixture of compounds in which the hydrophobic drug and lipophilic vehicle are soluble, and the porous substrate insoluble. The volatile solvent should also have relatively low toxicity and be removed from the composition to a level that is acceptable according to The International Committee on Harmonization (ICH) guidelines. Preferred volatile solvents include alcohols such as methanol, ethanol, n-propanol, isopropanol, and butanol; ketones such as acetone, methyl ethyl ketone and methyl iso-butyl ketone; esters such as ethyl acetate and propylacetate; and various other solvents such as acetonitrile, tetrahydrofuran, methylene chloride, toluene, and 1,1,1-trichloroethane. Mixtures of solvents, such as 50% methanol and 50% acetone, can also be used, as can mixtures with water. Preferred solvents include methanol, ethanol, acetone, methylene chloride, tetrahydrofuran, and mixtures thereof.

The hydrophobic drug and lipophilic vehicle are dissolved in the volatile solvent and the porous substrate is suspended in the volatile solvent to form a suspension or slurry. The volume of volatile solvent added may be any amount that facilitates adsorption and subsequent processing, but typically ranges from about 0.1 to 10 times the volume of the hydrophobic drug/lipophilic vehicle. Preferably the volume of volatile solvent used ranges from about 0.5 to about 4 times the combined volume of the hydrophobic drug/lipophilic vehicle.

It is preferred that the hydrophobic drug and lipophilic vehicle first be dissolved in the volatile solvent and then the porous substrate added to form a suspension or slurry, but this is not necessary for the practice of the invention. Without wishing to be bound by any particular theory or mechanism of action, it is believed that use of a volatile solvent sufficiently reduces the viscosity of the hydrophobic drug/lipophilic vehicle combination to facilitate penetration of the combination into the pores of the porous substrate, resulting in a higher loading of the hydrophobic drug/lipophilic vehicle on the porous substrate.

Once the hydrophobic drug, lipophilic vehicle, and porous substrate have been combined with the volatile solvent to form a suspension or slurry, it may be agitated to ensure the porous substrate is in the form of small particles. Agitation may be performed by any method that is capable of imparting sufficient energy to the suspension or slurry to break up agglomerations of porous substrate particles. Exemplary methods include overhead mixers, magnetically driven mixers and stir bars, planetary mixers, homogenizers, high speed mixing, high shear mechanical mixing, twin-screw mixing, single screw or twin-screw extruders, and the like. Sonication of the suspension or slurry may also be used to reduce agglomeration. The suspension or slurry may also be continuously agitated during processing to reduce agglomeration during processing.

At least a portion of the volatile solvent is removed from the suspension or slurry to form the solid compositions of the present invention. By “at least a portion” is meant that a sufficient amount of the volatile solvent is removed so that the suspension or slurry becomes a solid composition. Typically, this will occur when the solvent content of the composition is less than about 30 wt %, more preferably less than about 20 wt %. Exemplary processes for removing the volatile solvent include filtration, spray drying, lyophilization, evaporation, vacuum drying, and tray drying. Generally, the solvent content of the solid composition should be less than about 10 wt % and preferably less than about 2 wt %.

In one embodiment, the solid adsorbate of the present invention is a solid free-flowing powder. By “solid free-flowing powder” is meant that in an angle of repose test, the powder has an angle of repose of less than about 42 degrees. Preferably, the angle of repose is less than about 40 degrees. In a typical angle of repose test, the material is poured in a conical heap onto a level, flat horizontal surface and the angle formed with the horizontal is the angle of repose. See for example, Remington: The Science and Practice of Pharmacy, 20^{th} Edition (2000), and The Theory And Practice Of Industrial Pharmacy, by Lachman, Lieberman and Kanig (Lea and Febiger, publishers, 3^{rd} ed. 1986), hereby incorporated by reference herein.

The resulting solid adsorbates of hydrophobic drug, lipophilic vehicle, and porous substrate are solid materials. The hydrophobic drug is present in a sufficient amount to be pharmaceutically effective. In one preferred embodiment, the solid adsorbates have a relatively high drug loading in which the hydrophobic drug constitutes at least about 2 wt %, more preferably at least about 3 wt %, more preferably at least about 5 wt %, and even more preferably at least about 10 wt % of the solid adsorbate. Such high drug loadings facilitate formation of solid dosage forms such as tablets, since the amount of excipient devoted to solubilizing the hydrophobic drug is sufficiently low to allow the use of other tableting excipients. In another preferred embodiment, the solid adsorbate is a solid material that is incorporated into a dosage form that is formed using compressive forces, such as a compressed tablet, pill, or caplet.

### Concentration-Enhancement

The solid adsorbates of the present invention provide concentration-enhancement in a use environment relative to a control composition. The term “concentration enhancement” means that the composition provides increased concentration of dissolved drug in an aqueous use environment relative to a control composition consisting of an equivalent amount of drug alone. The control composition consists of the crystalline form of the drug in it most thermodynamically stable form at ambient conditions (25° C. and 50% relative humidity). In cases where no crystalline form of the drug is known, unformulated amorphous drug may be substituted for crystalline drug.

As used herein, an “aqueous use environment” can be either the in vivo environment of the GI tract or the in vitro environment of a test solution, such as the PBS, MFD solution, or solution to model the fed state previously described. Concentration enhancement may be determined through either in vitro dissolution tests or through in vivo tests. It has been determined that enhanced drug concentration in in vitro dissolution tests in such in vitro test solutions provide good indicators of in vivo performance and bioavailability. In particular, a composition of the present invention may be dissolution-tested by adding it to an in vitro test solution and agitating to promote dissolution, or by performing a membrane-permeation test as described herein.

Several methods, such as an in vitro dissolution test or a membrane permeation test may be used to evaluate the concentration-enhancement provided by the compositions of the present invention. When tested using an in vitro dissolution test, the compositions of the present invention meet at least one, and preferably both, of the following conditions. The first condition is that the combination increases the maximum drug concentration (MDC) of drug in the in vitro dissolution test relative to the control composition consisting of an equivalent amount of drug alone. Preferably, a composition of the present invention, when dosed to an aqueous use environment, provides a maximum drug concentration (MDC) that is at least 1.25-fold the MDC provided by a control composition. In other words, if the MDC provided by the control composition is 100 μg/mL, then a composition of the present invention containing a concentration-enhancing polymer provides an MDC of at least 125 μg/mL. More preferably, the MDC of drug achieved with the compositions of the present invention are at least 2-fold, even more preferably at least 3-fold, and most preferably at least 5-fold that of the control composition.

The second condition is that the compositions of the present invention provide in an aqueous use environment a concentration versus time Area Under the Curve (AUC), for any period of at least 90 minutes between the time of introduction into the use environment and about 270 minutes following introduction to the use environment that is at least 1.25-fold that of the control composition. More preferably, the AUC in the aqueous use environment achieved with the compositions of the present invention are at least 2-fold, more preferably at least 3-fold, and most preferably at least 5-fold that of a control composition.

An in vitro test to evaluate enhanced drug concentration can be conducted by (1) administering with agitation a sufficient quantity of test composition (that is, the solid composition of the present invention) in a test medium, such that if all of the drug dissolved, the theoretical concentration of drug would exceed the equilibrium concentration of the drug by a factor of at least 2; (2) in a separate test, adding an appropriate amount of control composition to an equivalent amount of test medium; and (3) determining whether the measured MDC and/or AUC of the test composition in the test medium is at least 1.25-fold that provided by the control composition. In conducting such a dissolution test, the amount of test composition or control composition used is an amount such that if all of the drug dissolved, the drug concentration would be at least 2-fold, preferably at least 10-fold, and most preferably at least 100-fold that of the aqueous solubility (that is, the equilibrium concentration) of the drug. For some test compositions containing a very low-solubility hydrophobic drug, it may be necessary to administer an even greater amount of the test composition to determine the MDC.

The concentration of dissolved drug is typically measured as a function of time by sampling the test medium and plotting drug concentration in the test medium vs. time so that the MDC and/or AUC can be ascertained. The MDC is taken to be the maximum value of dissolved drug measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than about 30 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred formulations. It is recognized that this definition of “dissolved drug” encompasses not only monomeric solvated drug molecules but also a wide range of species such as drug in micelles, emulsions, microemulsions, colloidal particles or nanoparticles, drug/oil or drug/surfactant aggregates, and other such drug-containing species that are present in the filtrate or supernatant in the specified dissolution test.

Alternatively, an in vitro membrane-permeation test may be used to evaluate the compositions of the present invention. In this test the composition is administered to an aqueous solution to form a feed solution. By “administered” is meant that the composition is placed in, dissolved in, suspended in, or otherwise delivered to the aqueous solution. The aqueous solution can be any physiologically relevant solution, as described above. After forming the feed solution, the solution may be agitated to dissolve or disperse the composition therein or may be added immediately to a feed solution reservoir. Alternatively, the feed solution may be prepared directly in a feed solution reservoir. Preferably, the feed solution is not filtered or centrifuged after administration of the pharmaceutical composition prior to performing the membrane-permeation test.

The feed solution is then placed in contact with the feed side of a microporous membrane, the feed side surface of the microporous membrane being hydrophilic. The portion of the pores of the membrane that are not hydrophilic are filled with an organic fluid, such as a mixture of decanol and decane, and the permeate side of the membrane is in fluid communication with a permeate solution comprising the organic fluid. Both the feed solution and the organic fluid remain in contact with the microporous membrane for the duration of the test. The length of the test may range from several minutes to several hours or even days.

The rate of transport of drug from the feed solution to the permeate solution is determined by measuring the concentration of drug in the organic fluid in the permeate solution as a function of time or by measuring the concentration of drug in the feed solution as a function of time, or both. This can be accomplished by methods well known in the art, including by use of ultraviolet/visible (UV/V is) spectroscopic analysis, high-performance liquid chromatography (HPLC), gas chromatography (GC), nuclear magnetic resonance (NMR), infra red (IR) spectroscopic analysis, polarized light, density, and refractive index. The concentration of drug in the organic fluid can be determined by sampling the organic fluid at discrete time points and analyzing for drug concentration or by continuously analyzing the concentration of drug in the organic fluid. For continuous analysis, UV/Vis probes may be used, as can flow-through cells. In all cases, the concentration of drug in the organic fluid is determined by comparing the results against a set of standards, as well known in the art.

From these data, the maximum flux of drug across the membrane is calculated by multiplying the slope of the concentration of drug in the permeate solution versus time plot by the permeate volume and dividing by the membrane area. This slope is typically determined during the initial portion of the test, where the concentration of drug in the permeate solution often increases at a nearly constant rate. At longer times, as more of the drug is removed from the feed solution, the slope of the concentration versus time plot decreases, becoming non-linear. Often, this slope approaches zero as the driving force for transport of drug across the membrane approaches zero; that is, the drug in the two phases approaches equilibrium. The maximum flux is determined either frorn the linear portion of the concentration versus time plot, or is estimated from a tangent to the concentration versus time plot at time equals zero if the curve is non-linear. Further details of this membrane-permeation test are presented in co-pending U.S. Patent Application Ser. No. 60/557,897, entitled “Method and Device for Evaluation of Pharmaceutical Compositions,” filed Mar. 30, 2004, (attorney Docket No. PC25968), incorporated herein by reference.

An in vitro membrane-permeation test to evaluate enhanced drug concentration can be conducted by (1) administering a sufficient quantity of test composition (that is, the solid composition of the present invention) to a feed solution, such that if all of the drug dissolved, the theoretical concentration of drug would exceed the equilibrium concentration of the drug by a factor of at least 2; (2) in a separate test, adding an equivalent amount of control composition to an equivalent amount of test medium; and (3) determining whether the measured maximum flux of drug provided by the test composition is at least 1.25-fold that provided by the control composition. A composition of the present invention provides concentration enhancement if, when dosed to an aqueous use environment, it provides a maximum flux of drug in the above test that is at least about 1.25-fold the maximum flux provided by the control composition. Preferably, the maximum flux provided by the compositions of the present invention are at least about 1.5-fold, more preferably at least about 2-fold, and even more preferably at least about 3-fold that provided by the control composition.

Alternatively, the compositions of the present invention, when dosed orally to a human or other animal, provide an AUC in drug concentration in the blood plasma or serum that is at least 1.25-fold that observed when an appropriate control composition is dosed. Preferably, the blood AUC is at least about 2-fold, preferably at least about 3-fold, preferably at least about 4-fold, preferably at least about 6-fold, preferably at least about 10-fold, and even more preferably at least about 20-fold that of the control composition. It is noted that such compositions can also be said to have a relative bioavailability of from about 1.25-fold to about 20-fold that of the control composition.

Alternatively, the compositions of the present invention, when dosed orally to a human or other animal, provide maximum drug concentration in the blood plasma or serum (Cₘₐₓ) that is at least 1.25-fold that observed when an appropriate control composition is dosed. Preferably, the blood Cₘₐₓ is at least about 2-fold, preferably at least about 3-fold, preferably at least about 4-fold, preferably at least about 6-fold, preferably at least about 10-fold, and even more preferably at least about 20-fold that of the control composition.

Relative bioavailability and Cₘₐₓ of drugs in the compositions can be tested in vivo in animals or humans using conventional methods for making such a determination. An in vivo test, such as a crossover study, may be used to determine whether the compositions of the present invention provide an enhanced relative bioavailability or Cₘₐₓ compared with a control composition as described above. In an in vivo crossover study a test composition comprising a composition of the present invention is dosed to half a group of test subjects and, after an appropriate washout period (e.g., one week) the same subjects are dosed with a control composition that consists of an equivalent quantity of crystalline drug as the test composition. The other half of the group is dosed with the control composition first, followed by the test composition. The relative bioavailability is measured as the concentration of drug in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC and Cₘₐₓ can be made by plotting the serum or plasma concentration of drug along the ordinate (y-axis) against time along the abscissa (x-axis). To facilitate dosing, a dosing vehicle may be used to administer the dose. The dosing vehicle is preferably water, but may also contain materials for suspending the test or control composition, provided these materials do not dissolve the composition or change the aqueous solubility of the drug in vivo. The determination of AUCs and Cₘₐₓ is a well-known procedure and is described, for example, in Welling, “Pharmacokinetics Processes and Mathematics,” ACS Monograph 185 (1986).

The compositions that, when evaluated, meet either the in vitro or the in vivo, or both, performance criteria are considered a part of this invention.

### Excipients and Dosage Forms

The solid adsorbates of the present invention comprising a hydrophobic drug, a lipophilic vehicle, and a porous substrate, may be formulated into solid dosage forms using procedures well known in the art. The inclusion of conventional excipients may be employed in the compositions of this invention, including those excipients well known in the art (see for example, Remington: The Science and Practice of Pharmacy, 20^{th} Edition (2000)). Generally, excipients such as fillers, disintegrating agents, pH modifiers such as acids, bases, or buffers, pigments, binders, lubricants, glidants, flavorants, and so forth may be used for customary purposes and in typical amounts without adversely affecting the properties of the compositions.

One method for forming the solid dosage form is to first blend the solid adsorbate of the invention with optional excipients using procedures well-known in the art. See for example, Remington: The Science and Practice of Pharmacy, 20^{th} Edition (2000). Examples of blending equipment include twin-shell blenders, fluidized beds, and V blenders. The blend may then be formulated using conventional procedures and equipment into solid oral dosage forms such as tablets, caplets, capsules that can contain the drug in the form of minitablets, beads, granules, pellets or other multiparticulates, pills, or powder. Compositions of this invention may be administered as immediate release, controlled release, delayed release, or chewable dosage forms, using procedures well known in the art.

Examples of matrix materials, fillers, or diluents include lactose, mannitol, xylitol, microcrystalline cellulose, dibasic calcium phosphate (dihydrate and anhydrous), and starch.

Examples of disintegrants include sodium starch glycolate, sodium alginate, carboxy methyl cellulose sodium, methyl cellulose, and croscarmellose sodium, and crosslinked forms of polyvinyl pyrrolidone such as those sold under the trade name CROSPOVIDONE (available from BASF Corporation).

Examples of binders include methyl cellulose, microcrystalline cellulose, starch, and gums such as guar gum, and tragacanth.

Examples of lubricants include magnesium stearate, calcium stearate, and stearic acid.

Examples of preservatives include sulfites (an antioxidant), benzalkonium chloride, methyl paraben, propyl paraben, benzyl alcohol and sodium benzoate.

Examples of suspending agents or thickeners include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, and titanium dioxide.

Examples of anticaking agents or fillers include silicon oxide and lactose.

In some cases, the overall dosage form or particles, granules or beads that make up the dosage form may have superior performance if coated with an enteric polymer to prevent or retard dissolution until the dosage form leaves the stomach. Exemplary enteric coating materials include HPMCAS, HPMCP, CAP, CAT, carboxymethylethyl cellulose, carboxylic acid-functionalized polymethacrylates, and carboxylic acid-functionalized polyacrylates.

In one preferred embodiment, the dosage form is a compressed dosage form, such as a compressed tablet, pill or caplet.

In one embodiment, the dosage form of the present invention, comprising an adsorbate of a CETP inhibitor, a lipophilic vehicle, and a porous substrate, further comprises an HMG-CoA reductase inhibitor, an important enzyme catalyzing the intracellular synthesis of cholesterol. Thus, a composition comprises (1) a solid adsorbate comprising a CETP inhibitor, a lipophilic vehicle, and a porous substrate, and (2) an HMG-CoA reductase inhibitor. In one aspect, the HMG-CoA reductase inhibitor is from a class of therapeutics commonly called statins. Preferably the HMG-CoA reductase inhibitor is selected from the group consisting of fluvastatin, lovastatin, pravastatin, atorvastatin, simvastatin, cerivastatin, rivastatin, mevastatin, velostatin, compactin, dalvastatin, fluindostatin, rosuvastatin, pitivastatin, dihydrocompactin, and pharmaceutically acceptable forms thereof. By “pharmaceutically acceptable forms” is meant any pharmaceutically acceptable derivative or variation, including stereoisomers, stereoisomer mixtures, enantiomers, solvates, hydrates, isomorphs, polymorphs, salt forms and prodrugs. In one embodiment, the HMG-CoA reductase inhibitor is selected from the group consisting of atorvastatin, fluvastatin, lovastatin, pravastatin, simvastatin, rosuvastatin, and pharmaceutically acceptable forms thereof. In a more preferred embodiment, the HMG-CoA reductase inhibitor is selected from the group consisting of atorvastatin, the cyclized lactone form of atorvastatin, a 2-hydroxy, 3-hydroxy or 4-hydroxy derivative of such compounds, and pharmaceutically acceptable forms thereof. Even more preferably, the HMG-CoA reductase inhibitor is atorvastatin hemicalcium trihydrate.

The amount of CETP inhibitor and HMG-CoA reductase inhibitor present in the dosage form will vary depending on the desired dose for each compound, which in tum, depends on the potency of the compound and the condition being treated. For example, the desired dose for the CETP inhibitor torcetrapib ranges from 1 mg/day to 1000 mg/day, preferably 10 to 250 mg/day, more preferably 30 to 90 mg/day. For the HMG-CoA reductase inhibitor atorvastatin calcium, the dose ranges from 1 to 160 mg/day, preferably 2 to 80 mg/day. For the HMG-CoA reductase inhibitors lovastatin, pravastatin sodium, simvastatin, rosuvastatin calcium, and fluvastatin sodium, the dose ranges from 2 to 160 mg/day, preferably 10 to 80 mg/day. For the HMG-CoA reductase inhibitor cerivastatin sodium, the dose ranges from 0.05 to 1.2 mg/day, preferably 0.1 to 1.0 mg/day.

To form a dosage form, the solid adsorbate comprising the CETP inhibitor may be combined with an HMG-CoA reductase inhibitor and optional excipients. The combination may be blended or granulated, and then formed into a dosage form, such as a sachet, oral powder for constitution, tablet, caplet, pill, capsule, and the like, all well known in the art. See, for example, Remington: The Science and Practice of Pharmacy (20^{th} Edition, 2000).

Other features and embodiments of the invention will become apparent from the following examples that are given for illustration of the invention rather than for limiting its intended scope.

### EXAMPLES

### Example 1

### Formation of Solid Self-Emulsifying Hydrophobic Drug Composition

A liquid self-emulsifying composition was prepared by first forming a lipophilic vehicle containing 20 wt % Miglyol® 812 N (a 56% caprylic and 36% capric trialkyl glyceride, available from Condea Vista Inc.), 30 wt % triacetin, 20 wt % of the polyoxyethylene sorbitan fatty acid ester (Tween® 80), and 30 wt % Capmul® MCM (mono- and di-alkyl glycerides of capric and caprylic acid, available from Abitec Corp.). Next, to 7.0 mL of this lipophilic vehicle was added 3.03 g of the lipophilic drug [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, also known as torcetrapib. The resulting suspension was stirred at 600 rpm for 7 hours, then centrifuged for 5 min at 13,000 G to separate undissolved drug. The supernatant was recovered and then diluted 1:1 (vol:vol) with methanol.

To form the solid self-emulsifying formulation, calcium silicate (Zeopharm® 600, available from J. M. Huber Corp., Edison, N.J.), having a surface area of 300 m²/gm and an average particle size of 6 μm, was first dried 2 hours in a vacuum oven. Next, 8 mL of the methanol-diluted composition was added dropwise to 1.0 g calcium silicate while mixing with a spatula. The resulting slurry was placed in a vacuum desiccator and dried overnight, forming a flowable powder.

The potency of the composition of Example 1 was analyzed by high-performance liquid chromatography (HPLC) and found to be about 15 wt % (that is, the drug constituted 15 wt % of the solid adsorbate). Thus, the solid adsorbate consisted of about 15 wt % drug, about 50 wt % lipophilic vehicle, and about 35 wt % porous substrate.

### In Vitro Evaluation of Concentration Enhancement

The solid composition of Example 1 was evaluated in vitro using a microcentrifuge dissolution test as follows. For this test, a sufficient amount of material was added to a microcentrifuge test tube so that the concentration of torcetrapib would have been 1000 μg/mL, if all of the drug had dissolved. The test was run in duplicate. The tubes were placed in a 37° C. temperature-controlled chamber, and 1.8 mL PBS at pH 6.5 and 290 mOsm/kg was added to each respective tube. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by HPLC. The contents of each tube were mixed on the vortex mixer and allowed to stand undisturbed at 37° C. until the next sample was taken. Samples were collected at 4, 10, 20, 40, 90, and 1200 minutes. The results are shown in Table 1.

### CONTROL 1

Control 1 (C1) consisted of crystalline torcetrapib alone, and a sufficient amount of material was added so that the concentration of drug would have been 1000 μg/mL, if all of the drug had dissolved.

TABLE 1

| | Time | Concentration | AUC |
|---|---|---|---|
| Example | (min) | (μg/mL) | (min*μg/mL) |
| 1 | 0 | 0 | 0 |
| | 4 | 260 | 500 |
| | 10 | 330 | 2,300 |
| | 20 | 350 | 5,700 |
| | 40 | 380 | 13,000 |
| | 90 | 390 | 32,200 |
| | 1200 | 380 | 459,100 |
| Crystalline | 0 | 0 | 0 |
| torcetrapib | 4 | <1 | <2 |
| (C1) | 10 | <1 | <8 |
| | 20 | <1 | <18 |
| | 40 | <1 | <38 |
| | 90 | <1 | <88 |
| | 1200 | <1 | <1,200 |

The concentrations of drug obtained in these samples were used to determine the maximum drug concentration (“MDC₉₀”) and the area under the concentration-versus-time curve (“AUC₉₀”) during the initial ninety minutes. The results are shown in Table 2.

TABLE 2

| | MDC₉₀ | AUC₉₀ | C₁₂₀₀ |
|---|---|---|---|
| Example | (μg/mL) | (min*μg/mL) | (μg/mL) |
| 1 | 390 | 32,200 | 380 |
| C1 | <1 | <88 | <1 |

The results show that Example 1 provided concentration-enhancement relative to crystalline drug alone. The composition of Example 1 provided an MDC₉₀ that was at least 390-fold that provided by crystalline drug, and an AUC₉₀ that was at least 365-fold that provided by crystalline drug.

### Examples 2-7

The solid compositions of Examples 2-7 were made as described for Example 1, varying the composition of the lipophilic vehicle, the amount of methanol, and the type of solid substrate, as shown in Table 3. In all cases, 8 mL of the methanol-diluted drug/lipophilic vehicle solution was added to 1 gm of the porous substrate, and the resulting slurry placed in a vacuum desiccator overnight to form a flowable powder. The potencies of the compositions of Examples 2-7 were determined as described in Example 1.

TABLE 3

| | | Ratio of | | |
|---|---|---|---|---|
| | | Methanol | | |
| | | to Drug/ | | |
| | Lipophilic Vehicle | Lipophilic | | |
| Exam- | Composition | Vehicle | | Potency |
| ple | (wt %) | (vol:vol) | Solid Substrate | (wt %) |
| 1 | 20% Miglyol, | 1:1 | Calcium silicate | 15 |
| | 30% Triacetin, | | (Zeopharm 600) | |
| | 20% Tween 80, | | | |
| | 30% Capmul MCM | | | |
| 2 | 20% Miglyol, | 1:1 | Calcium silicate | 11 |
| | 30% Triacetin, | | (Zeopharm 600) | |
| | 20% Tween 80, | | | |
| | 30% Capmul MCM | | | |
| 3 | 20% Miglyol, | 2:5 | Calcium silicate | 11 |
| | 30% Triacetin, | | (Zeopharm 600) | |
| | 20% Tween 80, | | | |
| | 30% Capmul MCM | | | |
| 4 | 20% Miglyol, | 3:10 | Calcium silicate | 11 |
| | 30% Triacetin, | | (Zeopharm 600) | |
| | 20% Tween 80, | | | |
| | 30% Capmul MCM | | | |
| 5 | 20% Miglyol, | 1:1 | Silicon dioxide** | 11 |
| | 30% Triacetin, | | (Cab-O-Sil M-5P) | |
| | 20% Tween 80, | | | |
| | 30% Capmul MCM | | | |
| 6 | 20% Miglyol, | 1:1 | Calcium silicate | 5.4 |
| | 10% Triacetin, | | (Zeopharm 600) | |
| | 50% Cremaphor | | | |
| | RH40*, | | | |
| | 20% Capmul MCM | | | |
| 7 | 20% Miglyol, | 1:1 | Silicon dioxide | 5.3 |
| | 10% Triacetin, | | (Cab-O-Sil M-5P) | |
| | 50% Cremaphor | | | |
| | RH40*, | | | |
| | 20% Capmul MCM | | | |

*Cremaphor RH40, a polyethoxylated hydrogenated castor oil, available from BASF Corp.
**Cab-O-Sil M-5P, available from Cabot Corp., having a surface area of about 200 m²/g and an average particle length of about 0.2 to 0.3 μm.

### In Vitro Evaluation of Concentration Enhancement

The compositions of Examples 2-4 were evaluated in vitro using a microcentrifuge dissolution test as described for Example 1. The results are shown in Table 4.

TABLE 4

| | Time | Concentration | AUC |
|---|---|---|---|
| Example | (min) | (μg/mL) | (min*μg/mL) |
| 2 | 0 | 0 | 0 |
| | 4 | 160 | 300 |
| | 10 | 170 | 1,300 |
| | 20 | 190 | 3,100 |
| | 40 | 220 | 7,200 |
| | 90 | 230 | 18,400 |
| 3 | 0 | 0 | 0 |
| | 4 | 120 | 200 |
| | 10 | 150 | 1,100 |
| | 20 | 170 | 2,700 |
| | 40 | 190 | 6,300 |
| | 90 | 200 | 16,000 |
| 4 | 0 | 0 | 0 |
| | 4 | 130 | 300 |
| | 10 | 190 | 1,200 |
| | 20 | 200 | 3,100 |
| | 40 | 220 | 7,200 |
| | 90 | 220 | 18,200 |

The concentrations of drug obtained in these samples were used to determine MDC₉₀ and AUC₉₀ during the initial ninety minutes. The results are shown in Table 5. Control 1 (C1) is shown again for comparison.

TABLE 5

| | MDC₉₀ | AUC₉₀ |
|---|---|---|
| Example | (μg/mL) | (min*μg/mL) |
| 2 | 230 | 18,400 |
| 3 | 200 | 16,000 |
| 4 | 220 | 18,200 |
| C1 | <1 | <88 |

The results show that Examples 2-4 provided concentration-enhancement relative to crystalline drug alone. The compositions of the present invention provided MDC₉₀ values that were at least from 200- to 230-fold that provided by crystalline drug, and AUC₉₀ values that were at least from 180- to 209-fold that provided by crystalline drug.

### Example 8

A liquid self-emulsifying composition was prepared by first forming a lipophilic vehicle containing 20 wt % Miglyol® 812 N, 30 wt % triacetin, 20 wt % Tween® 80, and 30 wt % Capmul® MCM. Next, to 7.0 mL of this lipophilic vehicle was added 3.0 g of the lipophilic drug torcetrapib. The resulting suspension was stirred at 600 rpm for 7 hours, then centrifuged for 5 min at 13,000 G to separate undissolved drug.

To form the solid self-emulsifying composition, the lipophilic vehicle/torcetrapib solution was added drop-wise to 0.5 g dried calcium silicate (Zeopharm® 600) while mixing with a spatula. A total of 1.4 mL solution was added to 0.5 g Zeopharm®, resulting in a free-flowing powder. Adding additional solution resulted in the formation of a sticky material that had poor flow characteristics. The solid composition was placed in a vacuum desiccator overnight.

The potency of the composition of Example 8 was determined using the procedures described for Example 1 to be 9.6 wt %.

### Example 9

A composition comprising a CETP inhibitor and an HMG-CoA reductase inhibitor is formed using the following procedure. First, a granulation of atorvastatin calcium was made using the following process. The granulation contained 13.9 wt % atorvastatin trihydrate hemicalcium salt, 42.4 wt % calcium carbonate (Pre-carb 150, available from Mutchler Inc., Westwood, N.J.), 17.7 wt % microcrystalline cellulose (Avicel PH 101, FMC Corp.), 3.8 wt % croscarmellose sodium (AcDiSol, FMC Corp.), 0.5 wt % polysorbate 80 (Crillet 4HP, Croda, Parsippany, N.J.), 2.6 wt % hydroxypropyl cellulose (Klucel E F, Hercules, Wilmington, Del.), and 19.2 wt % pregelatanized starch (Starch 1500, available from Colorcon, Inc., West Point, Pa.). To form the granulation, the atorvastatin calcium, calcium carbonate, microcrystalline cellulose, and starch were charged into a fluidized bed granulation apparatus. A granulating fluid comprising the polysorbate 80 and hydroxypropyl cellulose dissolved in water was sprayed into the fluidized material to form the granules. The weight of water used was equal to half the weight of the granulation. The granulation was then dried in the fluidized bed using air with an inlet temperature of about 45° C. until an end point of less than 2% water loss on drying was achieved. The granules were then milled using a Fitzpatrick M5A mill. The mill was fitted with a 0.03-inch rasping plate and a rasping bar operating at about 500 rpm in a knives forward direction (counter-clockwise). The average particle size of the granules was about 105 μm using screen analysis.

To form the composition of Example 9, the atorvastatin granulation is combined with the solid composition of Example 1. The amount of the atorvastatin granulation and the amount of the solid composition of Example 1 is adjusted such that the composition of Example 9 contains 60 mg of torcetrapib and 20 mgA of atorvastatin. The composition of Example 9 is then incorporated into a dosage form, such as by filling the material, together with optional excipients, into a capsule, or by blending the material with optional excipients and compressing the material into a tablet.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A solid adsorbate comprising:
(a) a hydrophobic drug having a Log P value of from about 4 to about 10;
(b) a water immiscible lipophilic vehicle, said lipophilic vehicle capable of forming a plurality of lipophilic droplets when administered to an aqueous use environment; and
(c) a porous substrate;
wherein said hydrophobic drug and said lipophilic vehicle are adsorbed onto said porous substrate, and wherein said hydrophobic drug constitutes at least about 2 wt % of said solid adsorbate.

2. The solid adsorbate of claim 1 wherein said hydrophobic drug has a Log P value of from about 4.5 to about 9, wherein said hydrophobic drug constitutes at least about 5 wt % of said adsorbate and said adsorbate is substantially free of water.

3. The solid adsorbate of claim 2 wherein said lipophilic vehicle comprises two or more materials selected from the group consisting of an oil, a surfactant, and a lipophilic solvent.

4. The solid adsorbate of claim 3 wherein said lipophilic vehicle comprises two or more materials selected from the group consisting of mono- and diglycerides of capric and caprylic acid, fractionated coconut oil, light vegetable oils, triacetin, soybean oil, safflower oil, corn oil, olive oil, cottonseed oil, arachis oil, sunflower seed oil, palm oil, rapeseed oil, stearyl alcohol, cetyl alcohol, cetostearyl alcohol, stearic acid, polyoxyethylene 6 apricot kernel oil, polyoxyethylene corn oil, propylene glycol monolaurate, propylene glycol dicaprylate/caprate, polyglyceryl oleate, sodium 1,4-bis(2-ethylhexyl) sulfosuccinate (also known as docusate sodium), sodium lauryl sulfate (SLS), short-chain glyceryl monoalkylates, polyglycolized glycerides, mono- and dialkylate esters of polyols, polyoxyethylene 20 sorbitan monooleate, polyoxyethylene 20 sorbitan monolaurate, polyethylene (40 or 60) hydrogenated castor oil, polyoxyethylene (35) castor oil, polyethylene (60) hydrogenated castor oil, alpha tocopheryl polyethylene glycol 1000 succinate (Vitamin E TPGS), glyceryl PEG 8 caprylate/caprate, PEG 32 glyceryl laurate, propylene carbonate, dimethylisosorbide, ethyl lactate, N-methylpyrrolidones, transcutol, glycofurol, peppermint oil, 1,2-propylene glycol, and polyethylene glycols.

5. The solid adsorbate of claim 1 wherein said porous substrate is selected from the group consisting of calcium silicate and silicone dioxide.

6. The solid adsorbate of claim 1 wherein said hydrophobic drug is a cholesteryl ester transfer protein (CETP) inhibitor.

7. The solid adsorbate of claim 6 wherein said CETP inhibitor is selected from the group consisting of [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, [2R,4S]4-[acetyl-(3,5-bis-trifluoromethyl-benzyl)-amino]-2-ethyl-6-trifluorometriyl-3,4-dihydro-2H-quinoline-1-carboxylic acid isopropyl ester, [2R,4S]4-[(3,5-Bis-trifluoromethyl-benzyl)-methoxycarbonyl-amino]-2-ethyl-6-trifluoromethyl-3,4-dihydro-2H-quinoline-1-carboxylic acid isopropyl ester, and (2R)-3-[[3-(4-chloro-3-ethylphenoxy)phenyl][[3-(1, 1, 2,2-tetrafluoroethoxy)phenyl]methyl]amino]-1,1,1-trifluoro-2-propanol.

8. (canceled)

9. A dosage form comprising the solid adsorbate of claim 1.

10. The dosage form of claim 9 wherein said hydrophobic drug is a cholesteryl ester transfer protein (CETP) inhibitor.

11. (canceled)

12. The dosage form of claim 10 further comprising an HMG-CoA reductase inhibitor.

13. The dosage form of claim 12 wherein said HMG-CoA reductase inhibitor comprises a compound selected from the group consisting of atorvastatin, the cyclized lactone form of atorvastatin, a 2-hydroxy, 3-hydroxy or 4-hydroxy derivative of such compounds, and pharmaceutically acceptable forms thereof.

14. A method for forming the a solid adsorbate, comprising:
(a) forming a suspension or slurry comprising
(1) a hydrophobic drug having a Log P value of from about 4 to about 10,
(2) a water immiscible lipophilic vehicle, said lipophilic vehicle capable of forming a plurality of lipophilic droplets when administered to an aqueous use environment,
(3) a porous substrate, and
(4) a volatile solvent; and
(b) removing at least a portion of said volatile solvent from said suspension or slurry so as to form said solid adsorbate;
wherein said solid adsorbate comprises said hydrophobic drug and said lipophilic vehicle adsorbed to said porous substrate, and wherein said hydrophobic drug constitutes at least about 2 wt % of said solid adsorbate.

15. The method of claim 14 wherein the volume of said volatile solvent ranges from about 0.5 to about 4 times the combined volume of said hydrophobic drug and said lipophilic vehicle and the volatile solvent is selected from the group consisting of methanol, ethanol, acetone, methylene chloride, tetrahydrofuran and mixtures thereof.
